(19) United States
(12) Patent Application Publication
(10) Pub. No.: US 2024/0349995 A1
(43) Pub. Date: Oct. 24, 2024

(54) SYSTEMS AND METHODS OF CONTROLLING ENDOSCOPIC LIGHT OUTPUT

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Xuanye WANG, Reading, MA (US); Xu CHEN, Wayland, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/687,963

(22) PCT Filed: Sep. 28, 2022
(86) PCT No.: PCT/US2022/044993

§ 371 Date: Feb. 29, 2024

Related U.S. Application Data
(60) Provisional application No. 63/262,988, filed on Oct. 25, 2021.

(51) Int. Cl. A61B 1/06; A61B 1/00

(52) U.S. Cl. CPC A61B 1/0655; A61B 1/000096

(57) ABSTRACT

Controlling endoscopic light output. One example is a method of operating an endoscopic system, the method comprising: providing, from an endoscopic console, light to an endoscope at a first illumination level; receiving, by the endoscopic console, a first electronic image from a camera head associated with the endoscope; partitioning, by the endoscopic console, the first electronic image into a plurality of regions; calculating, by the endoscopic console, a value indicative of exposure for each region of the plurality of regions, thereby creating a plurality of values indicative of exposure; determining, by the endoscopic console, that a distal end of the endoscope is outside a body cavity, the determination based on the plurality of values indicative of exposure; and reducing, by the endoscopic console, the light provided to the endoscope to a second illumination level lower than the first illumination level.

### CROSS-REFERENCE TO RELATED APPLICATIONS

This application claims the benefit of U.S. provisional application No. 63/262,988 filed Oct. 25, 2021 titled, “Systems and Methods of Controlling Endoscopic Light Output.” The provisional application is incorporated by reference herein as if reproduced in full below.

### BACKGROUND

During endoscopic surgical procedures, an endoscope (e.g., laparoscope, arthroscope) is provided light from a light source providing by way of a light guide. When an endoscope is not in use during the surgical procedure, the endoscope may be removed from the joint or the light guide may be un-plugged from the endoscope. In either case, high-intensity light may then shine into the surgical room from the endoscope's distal tip or the end of the light guide. The high-intensity light may cause thermal and over-illumination hazards, such as damage to retinas of the surgical team, or potentially scorching cloth and draping material of the surgical procedure which increases the fire potential.

### SUMMARY

One example is a method of operating an endoscopic system, the method comprising: providing, from an endoscopic console, light to an endoscope at a first illumination level; receiving, by the endoscopic console, a first electronic image from a camera head associated with the endoscope; partitioning, by the endoscopic console, the first electronic image into a plurality of regions; calculating, by the endoscopic console, a value indicative of exposure for each region of the plurality of regions, thereby creating a plurality of values indicative of exposure; determining, by the endoscopic console, that a distal end of the endoscope is outside a body cavity, the determination based on the plurality of values indicative of exposure; and reducing, by the endoscopic console, the light provided to the endoscope to a second illumination level lower than the first illumination level.

In the example method, reducing the light provided to the endoscope may further comprise reducing to the second illumination level being non-zero. Reducing to the second illumination level may further comprise reducing to between and including 1% and 10% of a total available light output of the endoscopic console. Reducing to the second illumination level may further comprise reducing to between and including 3% and 5% of a total available light output of the endoscopic console.

In the example method, determining that the distal end of the endoscope is outside the body cavity may further comprise: counting a number of regions of the plurality of regions having values indicative of exposure below a predetermined exposure threshold; and ascertaining that the distal end of the endoscope is outside the body cavity when the number is above a predetermined region threshold.

In the example method, determining that the distal end of the endoscope is outside the body cavity may further comprise: counting a number of regions of the plurality of regions having values indicative of exposure below a predetermined exposure threshold; reading an electronic gain value associated with displaying the first electronic image on a display device; and ascertaining that the distal end of the endoscope is outside the body cavity when the number is above a predetermined region threshold and the electronic gain value is above a predetermined gain threshold. The electronic gain value may be between 3 decibels (dB) and 6 dB inclusive.

In the example method, determining that the distal end of the endoscope is outside the body cavity may further comprise: counting a number of regions of the plurality of regions having values indicative of exposure below a predetermined exposure threshold; calculating an average acceleration over a period of time before receiving the first electronic image; and ascertaining that the distal end of the endoscope is outside the body cavity when the number is above a predetermined region threshold and the average acceleration is below a predetermined movement threshold.

In the example method, determining that the distal end of the endoscope is outside the body cavity may further comprise: counting a number of regions of the plurality of regions having values indicative of exposure below a predetermined exposure threshold; creating, by an artificial intelligence module of the endoscopic console, a scene indicator that a scene of the first electronic image is outside the body cavity; and ascertaining that the distal end of the endoscope is outside the body cavity when the number is above a predetermined region threshold and the scene indicator indicates the first electronic image is outside the body cavity.

The example method may further comprise, after reducing the light provided to the endoscope: receiving, by the endoscopic console, a second electronic image from the camera head; partitioning, by the endoscopic console, the second electronic image into a plurality of regions; calculating, by the endoscopic console, a second value indicative of exposure for each region of the plurality of regions of the second electronic image, thereby creating a second plurality of values indicative of exposure; determining, by the endoscopic console, that the distal end of the endoscope is within the body cavity, the determination based on the second plurality of values indicative of exposure; and increasing, by the endoscopic console, the light provided to the endoscope to a third illumination level higher than the second illumination level.

Another example is an endoscopic console comprising: a light port accessible on an outside surface of the endoscopic console, the light port configured to couple an endoscope by way of a light guide; a camera port accessible on an outside surface of the endoscopic console, the camera port configured to couple to a camera head and receive electronic images created by the camera head; light source optically coupled to the light port; and a console controller coupled camera port and the light source. The console controller may be configured to: command the light source to provide light to the light port at a first illumination level; receive a first electronic image from the camera head through the camera port; partition the first electronic image into a plurality of regions, and calculate a value indicative of exposure for each region of the plurality of regions, thereby creating a plurality of values indicative of exposure; determine that a distal end of an endoscope is outside a body cavity, the determination based on the plurality of values indicative of exposure; and reduce the light provided to the light port by commanding the light source to provide light at a second illumination level lower than the first illumination level.

In the example endoscopic console, when the console controller reduces the light provided to the light port, the console controller may be further configured to reduce to the second illumination level being non-zero. When the console controller reduces to the second illumination level, the console controller may be further configured to at least one selected from a group comprising: reduce to between and including 1% and 10% of a total available light output of the endoscopic console; and reduce to between and including 3% and 5% of a total available light output of the endoscopic console.

In the example endoscopic console, when the console controller determines that the distal end of the endoscope is outside the body cavity, the console controller may be further configured to: count a number of regions of the plurality of regions having values indicative of exposure below a predetermined exposure threshold; and ascertain that the distal end of the endoscope is outside the body cavity when the number is above a predetermined region threshold.

In the example endoscopic console, when the console controller determines that the distal end of the endoscope is outside the body cavity, the console controller may be further configured to: count a number of regions of the plurality of regions having values indicative of exposure below a predetermined exposure threshold; read an electronic gain value associated with displaying the first electronic image on a display device; and ascertain that the distal end of the endoscope is outside the body cavity when the number is above a predetermined region threshold and the electronic gain value is above a predetermined gain threshold. The gain electronic value may be between 3 decibels (dB) and 6 dB inclusive.

In the example endoscopic console, when the console controller determines that the distal end of the endoscope is outside the body cavity, the console controller may be further configured to: count a number of regions of the plurality of regions having values indicative of exposure below a predetermined exposure threshold; receive a plurality of acceleration values from an accelerometer of the camera head; calculate an average acceleration over a period of time before receiving the first electronic image; and ascertain that the distal end of the endoscope is outside the body cavity when the number is above a predetermined region threshold and the average acceleration is below a predetermined movement threshold.

In the example endoscopic console, when the console controller determines that the distal end of the endoscope is outside the body cavity, the console controller may be further configured to: count a number of regions of the plurality of regions having values indicative of exposure below a predetermined exposure threshold; create a scene indicator that a scene of the first electronic image is outside the body cavity; and ascertain that the distal end of the endoscope is outside the body cavity when the number is above a predetermined region threshold and the scene indicator indicates the first electronic image is outside the body cavity.

In the example endoscopic console, the console controller may be further configured to, after reduction of the light provided to the endoscope: receive a second electronic image from the camera head; partition the second electronic image into a plurality of regions; calculate a second value indicative of exposure for each region of the plurality of regions of the second electronic image, thereby creating a second plurality of values indicative of exposure; determine that the distal end of the endoscope is within the body cavity, the determination based on the second plurality of values indicative of exposure; and increase the light provided to the light port to a third illumination level higher than the second illumination level.

Another example is an endoscopic system comprising:
a display device; an endoscope comprising a light connector and a camera-head connector; a camera head coupled to the camera-head connector, the camera head configured to create electronic images; and an endoscopic console defining a light port coupled to the light connector of the endoscope, a camera port electrically coupled to the camera head, and a light source within the endoscopic console. The endoscopic console may performing any of the example above-noted tasks.

### BRIEF DESCRIPTION OF THE DRAWINGS

For a detailed description of example embodiments, reference will now be made to the accompanying drawings in which:

FIG. 1 shows an endoscopic system in accordance with at least some embodiments;

FIG. 2 shows a block diagram of an endoscopic console in accordance with at least some embodiments;

FIG. 3 shows an example image created by a camera head when an arthroscope is within a body cavity or joint space, in accordance with at least some embodiments;

FIG. 4 shows a flow diagram in accordance with at least some embodiments;

FIG. 5 shows a flow diagram in accordance with at least some embodiments;

FIG. 6 shows a flow diagram in accordance with at least some embodiments; and

FIG. 7 shows a flow diagram in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“Controller” shall mean, alone or in combination, individual circuit components, an application specific integrated circuit (ASIC), a microcontroller with controlling software, a reduced-instruction-set computing (RISC) with controlling software, a digital signal processor (DSP), a processor with controlling software, a programmable logic device (PLD), or a field programmable gate array (FPGA), configured to read inputs and drive outputs responsive to the inputs.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to systems and methods of controlling endoscopic light output. More particularly, examples are directed to determining, by an endoscopic console coupled to an endoscope, that the endoscope has been removed from a body cavity or joint space, and reducing the light output to reduce the potential for retinal damage and/or to reduce the chances of scorching cloth and draping material within the surgical room. More particularly still, in some examples a camera head coupled to the endoscope provides electronic images of a scene beyond the distal end of the endoscope, the electronic image provided to an endoscopic console. The endoscopic console in example systems makes a determination as to whether the endoscope has been removed from the body cavity or joint space by partitioning the electronic image into a plurality of regions, and calculating an exposure value for each of the plurality of regions. Based on the plurality of exposure values, the endoscopic console can determine that the endoscope has been removed from the body cavity or joint space, and the endoscopic console reduces the light intensity provided to the endoscope. In some examples, the reduction of light intensity is a reduction to a non-zero value (e.g., between 3% and 6% inclusive of total possible light output), such that the endoscopic console may also be able to determine that the endoscope has been reinserted into the body cavity or joint space and increase the light intensity provided to the endoscope. The specification turns to an example system to orient the reader.

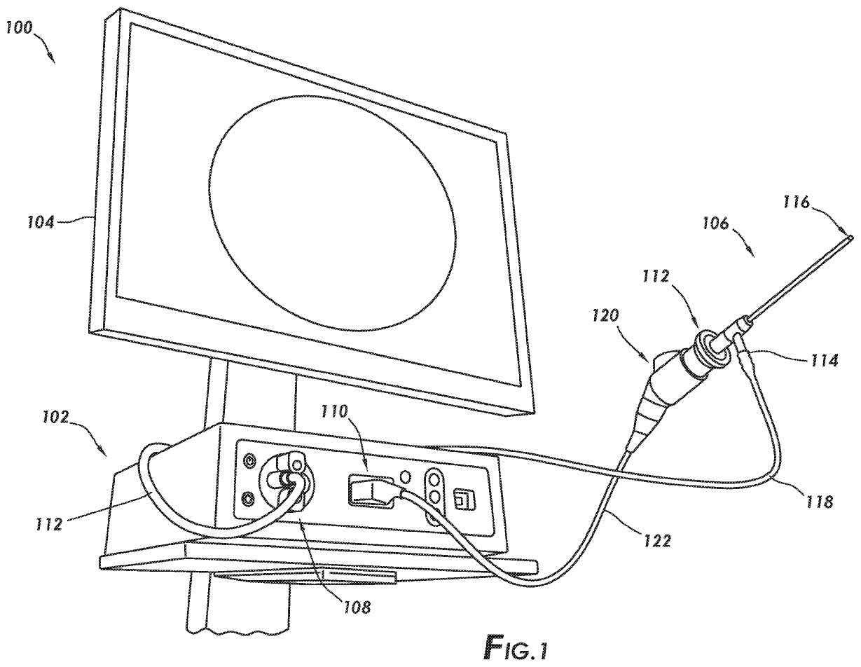

FIG. 1 shows an endoscopic system in accordance with at least some embodiments. In particular, FIG. 1 shows an endoscopic system 100 comprising an endoscopic console 102 coupled to a display device 104 and coupled to an endoscope 106 illustratively shown as an arthroscope (hereafter “arthroscope 106”). The endoscopic console 102 defines a light port 108 and a camera port 110, each port defined on an outside surface of the endoscopic console 102. The example arthroscope 106 defines a camera-head connector 112 on a proximal end, a light post 114, and a distal end 116 out which light shines and through which reflected light propagates back into the arthroscope 106. A light guide 118 optically couples between the light port 108 and the light post 114. In example cases, the light guide 118 may be one or more optical fibers designed and constructed to carry light from a light source within the endoscopic console 102 to the light port 108. A camera head 120 is mechanically and optically coupled to the camera-head connector 112 such that an optical array within the camera head 120 (e.g., charge-coupled device (CCD) array) captures images of tissue and structures beyond the distal end 116 of the arthroscope 106. The example camera head 120 is electrically coupled to the camera port 110 by way of an electrical cable 122, though any suitable communicative connection between the camera head 120 and the endoscopic console 102 may be used. The endoscopic console 102 may thus receive individual electronic images, or streams of electronics images in the form of video images, and display those images on the display device 104.

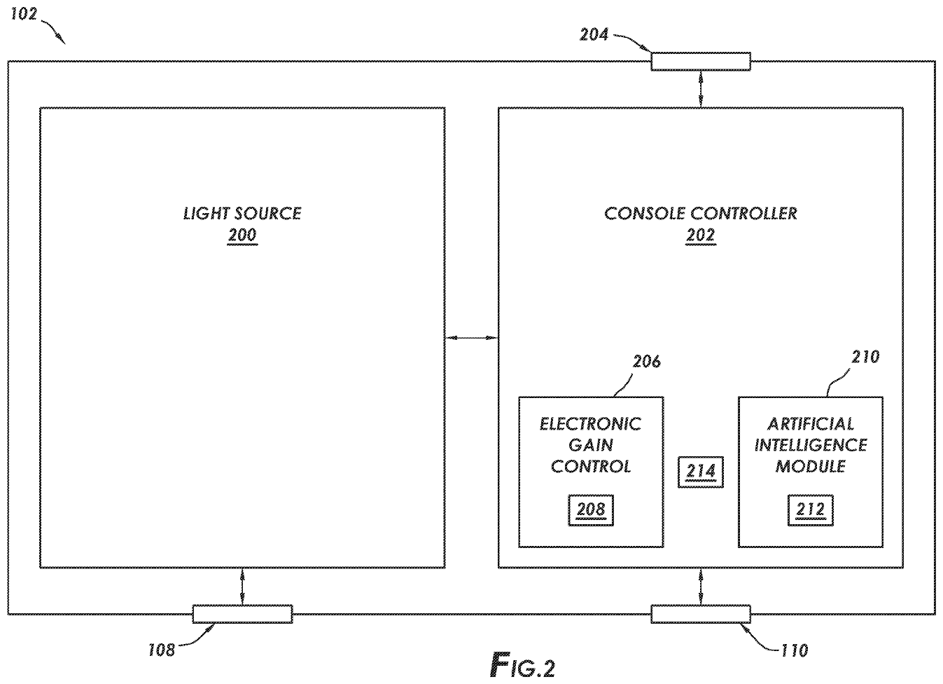

FIG. 2 shows a block diagram of an endoscopic console in accordance with at least some embodiments. In particular, FIG. 2 shows the endoscopic console 102 comprises the light port 108 and the camera port 110, each exposed on an outer surface of the endoscopic console 102. Additionally, the example endoscopic console 102 defines a display port 204 exposed on the outer surface of the endoscopic console 102, the display port 204 designed and constructed to operatively couple to a display device, such as display device 104 (FIG. 1). Internally, the example endoscopic console 102 comprises a light source 200 and a console controller 202. The example light source 200 may take any suitable form. In some cases, the light source 200 may comprise an incandescent bulb in combination with a moveable screen assembly with a varying width aperture. The intensity or illumination level of the light provided to the light port 108 may be controlled controlling the voltage provided to the incandescent bulb, controlling position of the moveable screen, or both. In yet still other cases, the light source may be a Xenon-based florescent bulb, again possibly in combination with a moveable screen assembly. The intensity of the light provided to the light port 108 when using a Xenon-based fluorescent bulb may be controlled by controlling the electrical energy (e.g., voltage, current, or both) provided to the xenon gas within the florescent bulb, controlling position of the moveable screen, or both. In yet still other cases, the light source may be a laser-light source, again possibly in combination with a moveable screen assembly. The intensity of the light provided to the light port 108 when using a laser-light source may be controlled by controlling the electrical energy (e.g., voltage, current, or both) provided to the laser-light source, controlling position of the moveable screen, or both. In yet still other cases, the light source 200 may be a series of light-emitting diodes (LEDs). The intensity of the light provided to the light port 108 when using LEDs may be controlled by controlling the average current through the LEDs. In summary, any suitable light source whose light intensity may be controlled electronically or mechanically may be used for the light source 200.

The console controller 202 is operatively coupled to the light source 200, communicatively coupled to the camera port 110, and communicatively coupled to the display port 204. The console controller 202 may take many suitable forms. In one example, the console controller 202 may be an application specific integrated circuit (ASIC) designed and constructed to perform the various methods discussed herein. In other cases, the console controller 202 may be a microcontroller with controlling software, along with various input devices and output devices, the controlling software designed and constructed to perform the various methods discussed herein. In further cases, the console controller 202 may be a processor, such as reduced-instruction-set computing (RISC), a digital signal processor (DSP), or a general purpose processor, along with controlling software designed and constructed to perform the various methods discussed herein. Further still, the console controller 202 may be a programmable logic device (PLD) or a field programmable gate array (FPGA) designed and constructed to perform the various methods described here. Yet further still, the console controller 202 may be implemented as combinations of any of the recited implementations. The specification now turns to an example explanation of determining when the arthroscope 106 has been removed from the body cavity or joint space.

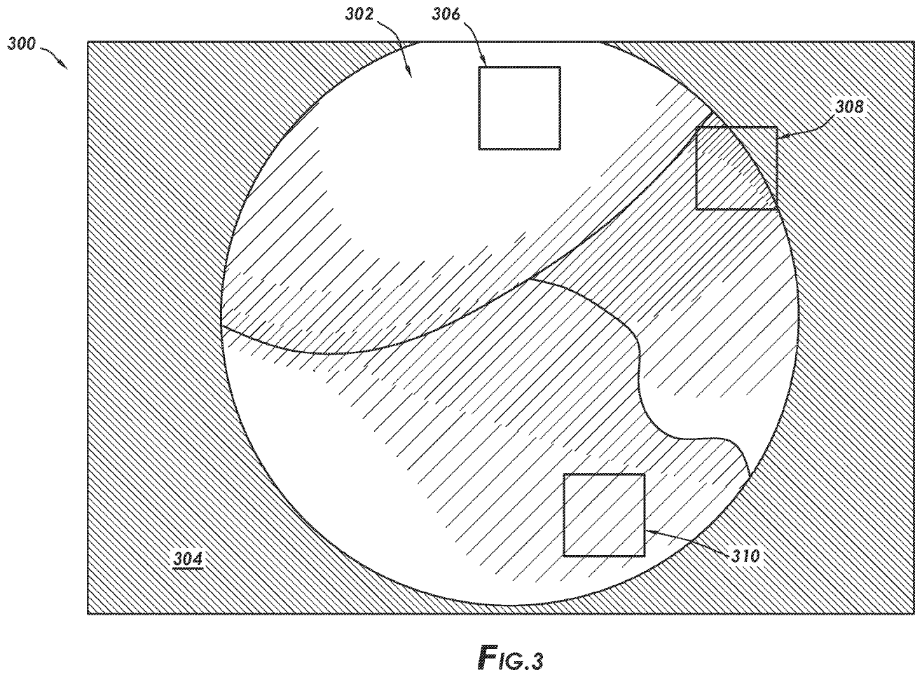

FIG. 3 shows an example image created by a camera head 120 (FIG. 1) when an arthroscope 106 (FIG. 1) is within a body cavity or joint space. In particular, FIG. 3 illustrates that the camera head 120 may produce a rectangular electronic image 300. The resolution of the electronic image 300 is based on the number of pixels of the optical array within the camera head 120. In one example the resolution of the electronic image is about 3840 pixels across or wide by about 2160 pixels up or tall, in shorthand notation “3840×2160.” The nomenclature for resolution is sometimes based on the number of horizontal pixels in the image, and thus an electronic image having 3840 horizontal pixels may be said to have about 4000 horizontal pixels, or a 4K resolution according to video display industry nomenclature. Many resolutions are possible for the camera head 120, such as lower resolutions and higher resolutions (e.g., 7680×4320 or 8K). In spite of the camera head 120 creating a rectangular electronic image 300, in most cases endoscopes capture a circular or oblong view of the tissue just beyond the distal end of the endoscope. Thus, the example electronic image 300 incudes image 302 bordered by unexposed or underexposed border area 304.

In example cases, each pixel of the electronic image has a color component. In some cases the color component is a multi-part value defining contribution of the three primary colors-red, green, and blue. For example, the color component may be 24 bits, with eight bits dedicated to red, eight bits dedicated to green, and eight bits dedicated to blue. Other color encoding schemes are also possible. The luminance of a pixel refers to the brightness of the pixel as perceived by the human eye. For electronic images encoded in color, the luminance may be a calculated value based on the combination of the red, green, and blue component values. For electronic images encoded in gray scale, the luminance value may be the value representing gray scale along a spectrum from black to stark white (e.g., 0 to 255 for an eight-bit encoding).

Regardless of the precise size of the electronic image and the color encoding scheme, in accordance with various examples a received electronic image is partitioned into a plurality of non-overlapping regions by the console controller 202 (FIG. 2). In example cases, each region is quadrilateral, such as a square or rectangle. However, in other cases, the regions may take any suitable form, and in fact the regions need not have a uniform shape. Further still, while in some cases the regions are non-overlapping, the various techniques work equally well with overlapping regions, such as when the overlap is uniformly distributed so as not to give undue weight to any particular sub-region.

In some example cases only regions that reside fully within the image 302 are used in the further determinations discussed below. In other words, the entire electronic image 300 may be partitioned, and any region that resides wholly or partially within the border area 304 may be omitted from the further determinations. In other cases, the entire electronic image 300 may be partitioned, and only regions that reside fully within the border area 304 are omitted, in which case regions that straddle the boundary of the border area 304 and the image 302 are considered in the further determinations. In yet still other cases, only regions that reside fully within the image 300 and regions that straddle the boundary of the border area 304 and the image 302 use, and regions that reside fully within the border area 304 are omitted.

Any suitable number of regions may be created by the partitioning. In some cases, as few as 20 regions may be used. In other cases, as many as 100 regions may be used. Some examples use between 40 and 80 regions, and in a particular example between 50 and 60 regions are used. While the entire electronic image 300 may be partitioned in some cases, and in other cases the just the image 302 may be partitioned, FIG. 3 shows only three regions 306, 308, and 310 so as not to unduly complicate the figure. Region 306 is an example of an over-exposed region, region 308 is an example of an under-exposed region (308), and region 310 is an example a correctly-exposed region (310). All the regions that reside within the border area 304, though no such regions are shown, would be considered unexposed regions or severely under-exposed regions.

In accordance with various examples, in order to determine whether the distal end of the arthroscope 106 (FIG. 1) is within or outside the body cavity or joint space, a value indicative of exposure is calculated for each region. Creating the value indicative of exposure for each region thereby creates a plurality of values indicative of exposure. Each value indicative of exposure can take any suitable form. For example, for electronic images encoded in a red, green, blue format, the value indicative of exposure may be an average or mean of the luminance values calculated from the individual color components for each pixel within the region. Stated otherwise, for each pixel within a region a luminance value is calculated, and the value indicative of exposure may be the mean or average of the luminance values. In cases where the electronic image is a gray-scale image, or is converted to a gray-scale image from color-encoded image, the value indicative of exposure may be a mean or average gray-scale value for each pixel within the region.

Once a value indicative of exposure is calculated for each region, the example method may then comprise counting a number of regions having a value indicative of exposure below a predetermined exposure threshold. Ascertaining that the distal end of the endoscope is outside the body cavity or joint space may occur when the number of regions is below a predetermined region threshold. That is to say, when the distal end of the arthroscope 106 is within the body cavity or joint space, the distal end of the arthroscope 106, may be just a few centimeters or less away from the various tissues of interest. It follows that when the distal end of the arthroscope 106 is within the body cavity or joint space, each region within the image 302 is likely to have a value indicative of exposure above the predetermined exposure threshold. The example electronic image of FIG. 3 is taken with the distal end of the arthroscope 106 within the body cavity or joint space, and thus the image 302 shows tissue and has a significant number of regions that are exposed (e.g., region 310 and related areas) and even over-exposed (region 306 and related areas). Even region 308, while being presented as comparatively underexposed, still would have a value indicative exposure well above any region from within the border area 304. By contrast, when the distal end of the arthroscope 106 is withdrawn from the body cavity or joint space, the closest light reflecting objects may be many meters away from distal end of the arthroscope 106, and thus less light is reflected back. It follows that a majority of the regions of an electronic image created with the arthroscope 106 withdrawn from the body cavity or joint space are likely to be underexposed. For example, when the distal end of the arthroscope 106 is removed from the body cavity or joint space and placed on a sterile tray, the image 302 may show a small portion of the tray (e.g., green sterile cloth draping), and the balance of electronic image will be out of focus and underexposed.

The examples discussed to this point calculate a value indicative of exposure, one each for each region, and then count the number of regions whose value indicative of exposure are below the predetermined exposure threshold. The opposite approach is also contemplated. That is, in other cases the counting may be with respect to regions whose value indicative of exposure are above the predetermined exposure threshold, and then ascertaining that the distal end of the arthroscope 106 is outside the body cavity or joint space when the number of regions is below the predetermined region threshold.

Returning briefly to FIG. 2, once a determination is made that the distal end of the arthroscope 106 (FIG. 1) has been removed from the body cavity or joint space, in accordance with example systems the console controller 202 reduces the light provided to the arthroscope 106. That is to say, when the distal end of the arthroscope 106 is within the body cavity or joint space, light at a certain illumination level is provided from the light source 200 to the arthroscope 106. However, once the distal end of the arthroscope 106 is removed from the body cavity or joint space, to reduce the chances of causing retinal damage to persons within the surgical room, and to reduce the chances of scorching cloth and draping material, in accordance with example embodiments the console controller 202 reduces the light provided to the arthroscope 106 to a lower illumination level. The console controller 202 reduces the light provided to the arthroscope 106 by communicating with the light source 200. The type of communication depends on the specific implementation of the light source 200. For example, when the light source is an incandescent bulb with a moveable screen, the console controller 202 may change a voltage setpoint of a power supply providing power to the incandescent bulb, may drive the moveable screen to a different position that blocks more of the light produced by the incandescent bulb, or both. In the case of the light source 200 being a string of LEDs, the console controller 202 may reduce the light provided to the endoscope by communicating a new, lower current setpoint to a current source providing power to the LEDs.

In some examples, the console controller 202 reduces the light provided to the endoscope to a non-zero level. That is, once the console controller 202 of the endoscopic console 102 determines the distal end of the arthroscope 106 has been removed from the body cavity or joint space, in example systems rather than cease all light provided to the arthroscope 106 (FIG. 1), the light is reduced an illumination level that is non-zero. The non-zero illumination level is selected to be low enough to reduce or eliminate the chances of causing retinal damage to persons within the surgical room and to reduce or eliminate the risk of scorching, but high enough that optical-based techniques may be used to determine that the distal end of the arthroscope 106 has been reinserted into the body cavity or joint space. In accordance with various examples, when a determination is made that the distal end of the arthroscope 106 has been removed from the body cavity or joint space, the console controller 202 reduces the light provided to the arthroscope to be 1% and 10% inclusive of the total possible light output of the light source 200, and in some cases to be between 3% and 5% inclusive of the total possible light output of the light source 200.

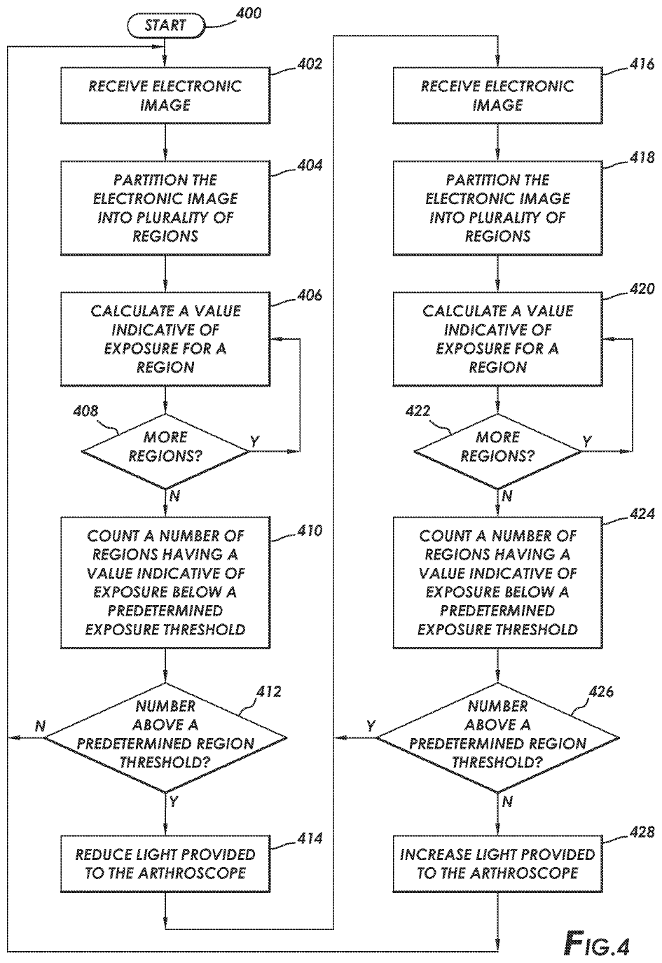

FIG. 4 shows a flow diagram of a method in accordance with at least some embodiments. The flow diagram of FIG. 4 may be implemented in whole or in part by software executing on a microcontroller or processor. In particular, the method starts (block 400) with an assumption that the distal end of the arthroscope 106 is disposed within the body cavity or joint space, and that the illumination level provided from the light source 200 (FIG. 2) of the endoscopic console 102 (FIG. 1) is a relatively high illumination level consistent with viewing the tissue within the body cavity or joint space. The example method then proceeds to receiving an electronic image (block 402). For example, the camera head 120 (FIG. 1) may capture an electronic image and transfer the electronic image to the console controller 202 (FIG. 2) of the endoscopic console 102 (FIG. 2). The next step in the illustrative flow diagram is the partitioning of the electronic image into a plurality of regions (block 404). For example, the console controller 202 may algorithmically perform the partitioning of the electronic image to create between 20 and 100 regions, in some cases between 40 and 80 regions, and in one specific example between 50 and 60 regions. Next, a value indicative of exposure is calculated for a region (block 406), and then a determination is made as to whether there are additional regions for which calculations are needed (block 408). The example method then loops between blocks 406 and 408 until all the regions have had a value indicative of exposure calculated. Once the values indicative of exposure are calculated, the next step in the example flow diagram is counting a number of regions having values indicative of exposure below predetermined exposure threshold (block 410), and then ascertaining whether the number of regions counted is above a predetermined region threshold (block 412). That is, if the number regions is above the predetermined region threshold (again block 412, the “Y” path), such is an indication that the distal end of the arthroscope 106 has been removed from the body cavity or joint space, and thus the example method reduces the light provided to the arthroscope (block 414). Oppositely, if the number of regions is below the predetermined region threshold (again block 412, the “N” path), such is an indication that the distal end of the arthroscope 106 is still within the body cavity or joint space, and thus the example method retreats to receiving the next electronic image (again block 402).

In some cases, the example method may end after ascertaining the distal end of the arthroscope 106 has been removed from the body cavity or joint space, and reducing the light provided to the arthroscope. However, in yet still further cases the optical-based method may be used in reverse to determine when the distal end of the arthroscope 106 has been reinserted. Still referring to FIG. 4, in example cases the method may then proceed to receiving another electronic image (block 416). The next step in the illustrative flow diagram is partitioning of the electronic image into a plurality of regions (block 418). Next, a value indicative of exposure is calculated for a region (block 420), and then a determination is made as to whether there are additional regions for which calculations are needed (block 422). The example method then loops between blocks 420 and 422 until all the values indicative of exposure are calculated. Once the values indicative of exposure are calculated, the next step in the example flow diagram is counting a number of regions having values indicative of exposure below the predetermined exposure threshold (block 424), and then ascertaining whether the number of regions counted is above a predetermined region threshold (block 426). That is, if the number of regions is above the predetermined region threshold (again block 426, the “Y” path), such is an indication that the distal end of the arthroscope 106 is still outside of the body cavity or joint space, and thus the example method retreats to receiving the next electronic image (again block 416). Oppositely, if the number of regions is below the predetermined region threshold (again block 426, the “N” path), such is an indication that the distal end of the arthroscope 106 has been reinserted, and thus the example method increase the light provided to the arthroscope (block 428) and once again the method begins looking for removal of the distal end of the arthroscope 106 from the body cavity or joint space by returning to block 402.

In the examples discussed to this point, the optical-based method is used alone to ascertain the status of the distal end of the arthroscope 106 as being within or outside the body cavity or joint space. However, in yet still further cases the determination may include additional, corroborating determinations, such as a corroborating electronic gain value associated with the displaying the electronic image, corroborating data from an accelerometer associated with the camera head, and/or corroborating scene indicators from an image recognition artificial intelligence. Each is discussed in turn.

Returning to FIG. 2, in example systems the console controller 202 further comprises and electronic gain control 206. In particular, the example console controller 202 receives electronic images, and controls the electronic gain applied when displaying the electronic images on the display device 104. That is, the electronic gain control 206 attempts to increase visibility of tissue within the image by increasing the gain when the electronic image may be slightly under-exposed, and the electronic gain control 206 also attempts to increase visibility of tissue structures within the image by decreasing the gain when the electronic image may be slightly over-exposed, washing out the fine detail. Note that the electronic gain control 206 does not change the amount of light provided to the camera head; rather, the electronic gain control 206 merely changes a gain parameter 208 applied to the electronic image prior to being sent through the display port 204 to the display device 104 (FIG. 1) for display.

In accordance with various examples, the gain parameter is used as corroboration when making a determination as to the state of the distal end of the arthroscope 106. In particular, in example cases determining that the distal end of the arthroscope is outside the body cavity comprises counting the number of regions having values indicative of exposure below the predetermined exposure threshold, reading the gain parameter value associated with the displaying of the electronic image on the display device 104 (FIG. 1), and ascertaining that the distal end of the arthroscope 106 is outside the body cavity or joint space when the number is above a predetermined region threshold and the electronic gain value is above a predetermined gain threshold. That is to say, if the electronic gain control 206 has significantly increased the gain parameter 208 in an effort to increase the perceived illumination in the electronic image displayed on the display device, then such corroborates that likely the distal end of the arthroscope 106 has been removed from the body cavity or joint space.

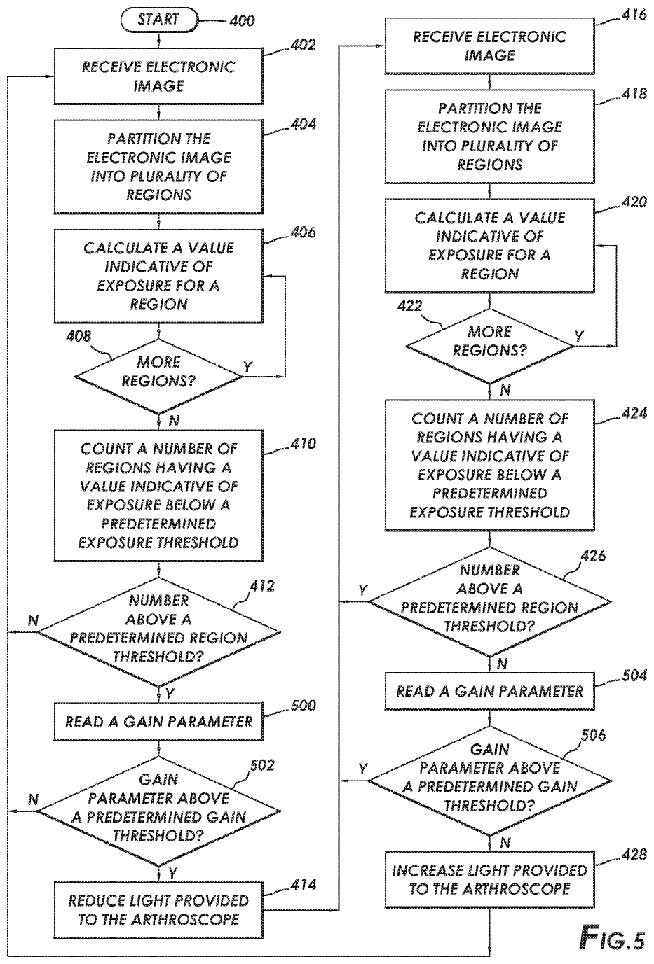

FIG. 5 shows a flow diagram of a method in accordance with at least some embodiments. The flow diagram of FIG. 5 may be implemented in whole or in part by software executing on a microcontroller or processor. Many of the example steps of FIG. 5 are duplicates of FIG. 4. The duplicate steps are labeled with duplicate reference numbers, and those steps will not be presented again so as not to unduly lengthen the specification. In the example method, if the number of regions is above the predetermined regions threshold (block 412), the next example step is reading the gain parameter (block 500). For example, the console controller 202 (FIG. 2) reads the gain parameter 208 (FIG. 2) from the electronic gain control 206 (FIG. 2). The next step in the example method is ascertaining whether the gain parameter is above a predetermined gain threshold (block 502). In one example, a gain value of 3 decibels (dB) or higher is used, and in one specific example a gain value of between 3 dB and 6 dB inclusive is used. If yes, then the light provided to the arthroscope 106 is reduced (block 414). If the gain parameter is below the predetermined gain threshold (again block 502), then the example method retreats to receiving the next electronic image (again block 402).

Similarly on the right hand side of FIG. 5 directed to optical-based determinations that the distal end of the arthroscope 106 has been reinserted into the body cavity or joint space, if the number of regions is not above the predetermined region threshold (block 426), the next example step is reading the gain parameter (block 504). Thereafter, the example method ascertains whether the gain parameter is above a predetermined gain threshold (block 506). If yes, then likely the distal end of the arthroscope 106 is still outside the body cavity or joint space, and thus the example method retreats to receiving the next electronic image (again block 416). On the other hand, if the gain parameter is below the predetermined threshold (again block 506), then likely the distal end of the arthroscope 106 has been reinserted into the body cavity or joint space, and thus the example method increases the light provided to the arthroscope (block 428), and once again the method begins looking for removal of the distal end of the arthroscope 106 from the body cavity or joint space by returning to block 402.

Returning to FIG. 2, in example systems the console controller 202 further comprises an artificial intelligence module 210. The artificial intelligence module 210 may take any suitable form, such as a multi-layer neural network trained with a data set of electronic images showing tissue as seen through an arthroscope 106 when the distal end of the arthroscope 106 is within a body cavity or joint space. In particular, in the example system the console controller 202 receives electronic images, and provides the electronic images to the artificial intelligence module 210. The artificial intelligence module 210, in turn, provides an output signal or scene indicator 212 that indicates whether the scene of the electronic image is recognized by the artificial intelligence module 210. That is, if the artificial intelligence module 210 indicates that the electronic image is recognized as containing tissue, then the distal end of the arthroscope 106 is likely within the body cavity or joint space. Oppositely, if the artificial intelligence module 210 indicates that the electronic image is not recognized as containing tissue, then the distal end of the arthroscope 106 is likely outside the body cavity or joint space.

In accordance with various examples, the scene indicator 212 is used as corroboration when making a determination as to the state of the distal end of the arthroscope 106. In particular, in example cases determining that the distal end of the arthroscope is outside the body cavity comprises counting the number of regions having values indicative of exposure below the predetermined exposure threshold, reading the scene indicator, and ascertaining that the distal end of the arthroscope 106 is outside the body cavity or joint space when the number is above a predetermined region threshold and the scene indicator is de-asserted. That is to say, if the scene indicator is de-asserted (e.g., scene not recognized), then such corroborates that likely the distal end of the arthroscope 106 has been removed from the body cavity or joint space.

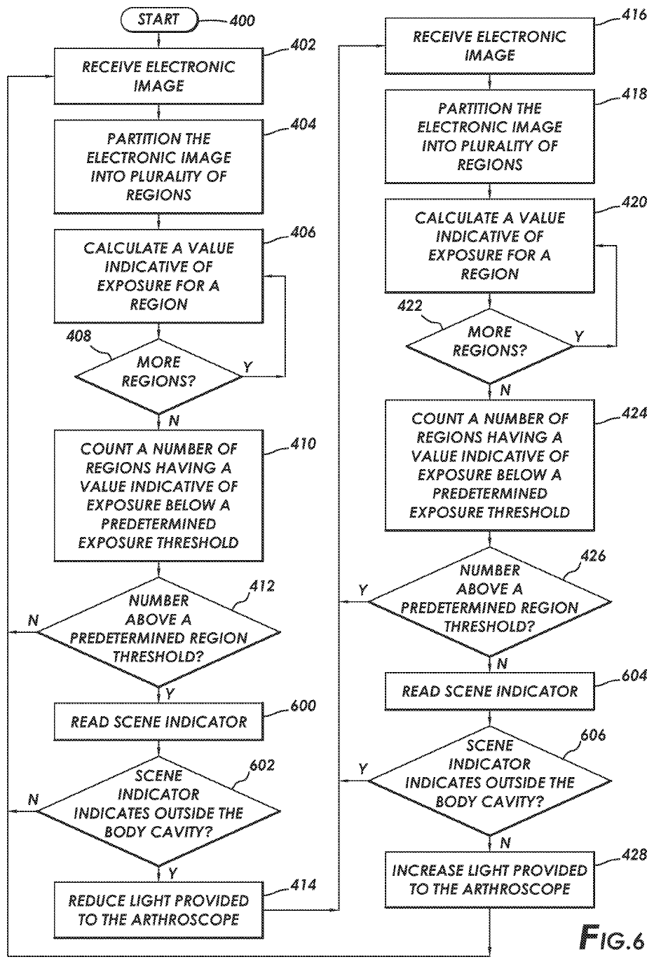

FIG. 6 shows a flow diagram of a method in accordance with at least some embodiments. The flow diagram of FIG. 6 may be implemented in whole or in part by software executing on a microcontroller or processor. Many of the example steps of FIG. 6 are duplicates of FIG. 4. The duplicate steps are labeled with duplicate reference numbers, and those steps will not be presented again so as not to unduly lengthen the specification. In the example method, if the number of regions is above the predetermined regions threshold (block 412), the next example step is reading the scene indicator (block 600). For example, the console controller 202 (FIG. 2) reads the scene indicator 212 (FIG. 2) from the artificial intelligence module 210 (FIG. 2). The method then ascertains whether the scene indicator indicates the electronic image is outside the body cavity (block 602). If yes, then the light provided to the arthroscope 106 is reduced (block 414). If the scene indicator indicates the scene of the electronic image is inside the body cavity or joint space, then the example method retreats to receiving the next electronic image (again block 402).

Similarly on the right hand side of FIG. 6 directed to optical-based determinations that the distal end of the arthroscope 106 has been reinserted back into the body cavity or joint space, if the number of regions is not above the predetermined region threshold (block 426), the next example step is reading the scene indicator (block 604). The method then ascertains whether the scene indicator indicates the electronic image is outside the body cavity (block 606). If yes, then likely the distal end of the arthroscope 106 is still outside the body cavity or joint space, and thus the example method retreats to receiving the next electronic image (again block 416). On the other hand, if the scene indicator indicates the electronic images is from inside the body cavity or joint space (again block 606), then likely the distal end of the arthroscope 106 has been reinserted into the body cavity or joint space, and thus the example method increases the light provided to the arthroscope (block 428), and once again the method begins looking for removal of the distal end of the arthroscope 106 from the body cavity or joint space by returning to block 402.

In yet still further example systems, the camera head 120 includes a movement sensor (e.g., tilt sensor and/or accelerometer) that produces values indicative of movement of the camera head 120. When the distal end of the arthroscope 106 resides within the body cavity or joint space, a certain amount of movement is expected. That is, the surgeon may be moving an appendage of the patient, and that movement is sensed by the movement sensor. Even if the surgeon is not physically moving the patient, movement of other surgical devices (e.g., mechanical resection instruments, ablation instruments), inserted through apertures through the patient's skin, cause slight movements of the patient that may be detected by the movement sensor of the camera head 120. Returning to FIG. 2, in example system the console controller 202 receives movement values from the camera head 120, calculates value indicative of movement, and places the resultant in the movement value 214. The value indicative of movement may take any suitable form, such as a mean or average over a predetermined period of time (e.g., last 30 seconds, last minute). If the movement value 214 is above a predetermined movement threshold, then the distal end of the arthroscope 106 is likely within the body cavity or joint space. Oppositely, if the movement value 214 is below the predetermined movement threshold, then the distal end of the arthroscope 106 is likely outside the body cavity or joint space.

In accordance with various examples, the movement value 214 is used as corroboration when making a determination as to the state of the distal end of the arthroscope 106. In particular, in example cases determining that the distal end of the arthroscope is outside the body cavity comprises counting the number of regions having values indicative of exposure below the predetermined exposure threshold, reading the movement value, and ascertaining that the distal end of the arthroscope 106 is outside the body cavity or joint space when the number is above a predetermined region threshold and the movement value is below the predetermined movement threshold (e.g., the arthroscope is laying the instrument tray). That is to say, if the movement value is below the predetermined threshold, then such corroborates that likely the distal end of the arthroscope 106 has been removed from the body cavity or joint space.

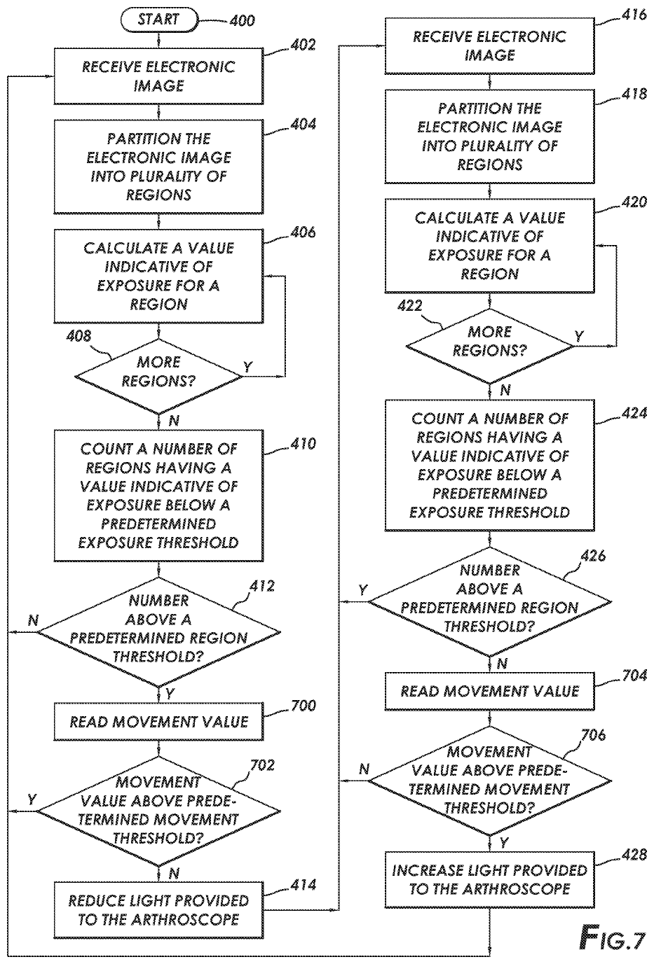

FIG. 7 shows a flow diagram of a method in accordance with at least some embodiments. The flow diagram of FIG. 7 may be implemented in whole or in part by software executing on a microcontroller or processor. Many of the example steps of FIG. 7 are duplicates of FIG. 4. The duplicate steps are labeled with duplicate reference numbers, and those steps will not be presented again so as not to unduly lengthen the specification. In the example method, if the number of regions is above the predetermined region threshold (block 412), the next example step is reading the movement value (block 700). For example, the console controller 202 (FIG. 2) reads the movement value 214 (FIG. 2). The next step in the example method is ascertaining whether the movement value is above the predetermined movement threshold (block 702). If yes, then the example method retreats to receiving the next electronic image (again block 402). On the other hand, if the movement value is below the predetermined movement threshold (again block 702), then the example method reduces the light provided to the arthroscope (block 414).

Similarly on the right hand side of FIG. 7 directed to optical-based determinations that the distal end of the arthroscope 106 has been reinserted back into the body cavity or joint space, if the number of regions is not above the predetermined region threshold (block 426), the next example step is reading the movement value (block 704). The example method then ascertains whether movement value is above the predetermined movement threshold (block 706). If no, then likely the distal end of the arthroscope 106 is still outside the body cavity or joint space, and thus the example method retreats to receiving the next electronic image (again block 416). On the other hand, if the movement value is above the predetermined movement threshold (again block 706), then likely the distal end of the arthroscope 106 has been reinserted into the body cavity or joint space, and thus the example method increases the light provided to the arthroscope (block 428), and once again the method begins looking for removal of the distal end of the arthroscope 106 from the body cavity or joint space by returning to block 402.

The various examples discussed to this point have been based on the corroborating information in a particular form. However, one of ordinary skill, with the benefit of this disclosure now understanding example systems and methods, could implement an equivalent system where the corroborating information takes a different form. For example, the analysis of the gain parameter (FIG. 5) could be an analysis of whether the gain parameter is below the predetermined gain threshold, with corresponding changes in the flow diagram. Further still, the example scene indicator of the artificial intelligence module 210 (FIG. 6) could be trained to recognize scenes outside the body, and the scene indicator 212 may be asserted when the scene indicates the distal end of the arthroscope 106 is outside body cavity or joint space, with corresponding changes in the flow diagram. Finally, the analysis of the movement value (FIG. 7) could be an analysis of whether the movement value is below the predetermined gain threshold, with corresponding changes in the flow diagram.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A method of operating an endoscopic system, the method comprising:
providing, from an endoscopic console, light to an endoscope at a first illumination level;
receiving, by the endoscopic console, a first electronic image from a camera head associated with the endoscope;
partitioning, by the endoscopic console, the first electronic image into a plurality of regions;
calculating, by the endoscopic console, a value indicative of exposure for each region of the plurality of regions, thereby creating a plurality of values indicative of exposure;
determining, by the endoscopic console, that a distal end of the endoscope is outside a body cavity, the determination based on the plurality of values indicative of exposure; and
reducing, by the endoscopic console, the light provided to the endoscope to a second illumination level lower than the first illumination level.

2. The method of claim 1 wherein reducing the light provided to the endoscope further comprises reducing to the second illumination level being non-zero.

3. The method of claim 2 wherein reducing to the second illumination level further comprises at least one selected from a group comprising. reducing to between and including 1% and 10% of a total available light output of the endoscopic console; and reducing to between and including 3% and 5% of a total available light output of the endoscopic console.

4. (canceled)

5. The method of claim 1 wherein determining that the distal end of the endoscope is outside the body cavity further comprises:
counting a number of regions of the plurality of regions having values indicative of exposure below a predetermined exposure threshold; and
ascertaining that the distal end of the endoscope is outside the body cavity when the number is above a predetermined region threshold.

6. The method of claim 1 wherein determining that the distal end of the endoscope is outside the body cavity further comprises:
counting a number of regions of the plurality of regions having values indicative of exposure below a predetermined exposure threshold;
reading an electronic gain value associated with displaying the first electronic image on a display device; and
ascertaining that the distal end of the endoscope is outside the body cavity when the number is above a predetermined region threshold and the electronic gain value is above a predetermined gain threshold.

7. (canceled)

8. The method of claim 1 wherein determining that the distal end of the endoscope is outside the body cavity further comprises:
counting a number of regions of the plurality of regions having values indicative of exposure below a predetermined exposure threshold;
calculating an average acceleration over a period of time before receiving the first electronic image; and
ascertaining that the distal end of the endoscope is outside the body cavity when the number is above a predetermined region threshold and the average acceleration is below a predetermined movement threshold.

9. The method of claim 1 wherein determining that the distal end of the endoscope is outside the body cavity further comprises:
counting a number of regions of the plurality of regions having values indicative of exposure below a predetermined exposure threshold;
creating, by an artificial intelligence module of the endoscopic console, a scene indicator that a scene of the first electronic image is outside the body cavity; and
ascertaining that the distal end of the endoscope is outside the body cavity when the number is above a predetermined region threshold and the scene indicator indicates the first electronic image is outside the body cavity.

10. The method of claim 1 further comprising, after reducing the light provided to the endoscope:
receiving, by the endoscopic console, a second electronic image from the camera head;
partitioning, by the endoscopic console, the second electronic image into a plurality of regions;
calculating, by the endoscopic console, a second value indicative of exposure for each region of the plurality of regions of the second electronic image, thereby creating a second plurality of values indicative of exposure;
determining, by the endoscopic console, that the distal end of the endoscope is within the body cavity, the determination based on the second plurality of values indicative of exposure; and
increasing, by the endoscopic console, the light provided to the endoscope to a third illumination level higher than the second illumination level.

11. An endoscopic console comprising:
a light port accessible on an outside surface of the endoscopic console, the light port configured to couple an endoscope by way of a light guide;
a camera port accessible on an outside surface of the endoscopic console, the camera port configured to couple to a camera head and receive electronic images created by the camera head;
light source optically coupled to the light port;
a console controller coupled camera port and the light source, the console controller configured to:
command the light source to provide light to the light port at a first illumination level;
receive a first electronic image from the camera head through the camera port;
partition the first electronic image into a plurality of regions, and calculate a value indicative of exposure for each region of the plurality of regions, thereby creating a plurality of values indicative of exposure;
determine that a distal end of an endoscope is outside a body cavity, the determination based on the plurality of values indicative of exposure; and
reduce the light provided to the light port by commanding the light source to provide light at a second illumination level lower than the first illumination level.

12. The endoscopic console of claim 11 wherein when the console controller reduces the light provided to the light port, the console controller is further configured to reduce to the second illumination level being non-zero.

13. The endoscopic console of claim 12 wherein when the console controller reduces to the second illumination level, the console controller is further configured to at least one selected from a group comprising: reduce to between and including 1% and 10% of a total available light output of the endoscopic console; and reduce to between and including 3% and 5% of a total available light output of the endoscopic console.

14. The endoscopic console of claim 11 wherein when the console controller determines that the distal end of the endoscope is outside the body cavity, the console controller is further configured to:
count a number of regions of the plurality of regions having values indicative of exposure below a predetermined exposure threshold; and
ascertain that the distal end of the endoscope is outside the body cavity when the number is above a predetermined region threshold.

15. The endoscopic console of claim 11 wherein when the console controller determines that the distal end of the endoscope is outside the body cavity, the console controller is further configured to:
count a number of regions of the plurality of regions having values indicative of exposure below a predetermined exposure threshold;
read an electronic gain value associated with displaying the first electronic image on a display device; and
ascertain that the distal end of the endoscope is outside the body cavity when the number is above a predetermined region threshold and the electronic gain value is above a predetermined gain threshold.

16. (canceled)

17. The endoscopic console of claim 11 wherein when the console controller determines that the distal end of the endoscope is outside the body cavity, the console controller is further configured to:
count a number of regions of the plurality of regions having values indicative of exposure below a predetermined exposure threshold;
receive a plurality of acceleration values from an accelerometer of the camera head;
calculate an average acceleration over a period of time before receiving the first electronic image; and
ascertain that the distal end of the endoscope is outside the body cavity when the number is above a predetermined region threshold and the average acceleration is below a predetermined movement threshold.

18. The endoscopic console of claim 11 wherein when the console controller determines that the distal end of the endoscope is outside the body cavity, the console controller is further configured to:
count a number of regions of the plurality of regions having values indicative of exposure below a predetermined exposure threshold;
create a scene indicator that a scene of the first electronic image is outside the body cavity; and
ascertain that the distal end of the endoscope is outside the body cavity when the number is above a predetermined region threshold and the scene indicator indicates the first electronic image is outside the body cavity.

19. The endoscopic console of claim 11 wherein the console controller is further configured to, after reduction of the light provided to the endoscope:
receive a second electronic image from the camera head;
partition the second electronic image into a plurality of regions;
calculate a second value indicative of exposure for each region of the plurality of regions of the second electronic image, thereby creating a second plurality of values indicative of exposure;
determine that the distal end of the endoscope is within the body cavity, the determination based on the second plurality of values indicative of exposure; and
increase the light provided to the light port to a third illumination level higher than the second illumination level.

20. An endoscopic system comprising:
a display device;
an endoscope comprising a light connector and a camera-head connector;
a camera head coupled to the camera-head connector, the camera head configured to create electronic images;
an endoscopic console defining a light port coupled to the light connector of the endoscope, a camera port electrically coupled to the camera head, and a light source within the endoscopic console, the endoscopic console configured to:
command the light source to provide light to the light connector at a first illumination level;
receive a first electronic image from the camera head;
partition the first electronic image into a plurality of regions, and calculate a value indicative of exposure for each region of the plurality of regions, thereby creating a plurality of values indicative of exposure;
determine that a distal end of the endoscope is outside a body cavity, the determination based on the plurality of values indicative of exposure; and
reduce the light provided to the light port by commanding the light source to provide light at a second illumination level lower than the first illumination level.

21. The endoscopic system of claim 20 wherein when the endoscopic console reduces the light from the light source, the console controller is further configured to reduce to the second illumination level being non-zero.

22. The endoscopic system of claim 21 wherein when the endoscopic console reduces to the second illumination level, the console controller is further configured to at least one selected from a group comprising: reduce to between and including 1% and 10% of a total available light output of the endoscopic console; and reduce to between and including 3% and 5% of a total available light output of the endoscopic console.

23. The endoscopic system of claim 20 wherein when the endoscopic console determines that the distal end of the endoscope is outside the body cavity, the endoscopic console is further configured to:
count a number of regions of the plurality of regions having values indicative of exposure below a predetermined exposure threshold; and
ascertain that the distal end of the endoscope is outside the body cavity when the number is above a predetermined region threshold.

24. The endoscopic system of claim 20 wherein when the endoscopic console determines that the distal end of the endoscope is outside the body cavity, the endoscopic console is further configured to:
count a number of regions of the plurality of regions having values indicative of exposure below a predetermined exposure threshold;
read an electronic gain value associated with displaying the first electronic image on a display device; and
ascertain that the distal end of the endoscope is outside the body cavity when the number is above a predetermined region threshold and the electronic gain value is above a predetermined gain threshold.

25. (canceled)

26. The endoscopic system of claim 20 wherein when the endoscopic console determines that the distal end of the endoscope is outside the body cavity, the endoscopic console is further configured to:
count a number of regions of the plurality of regions having values indicative of exposure below a predetermined exposure threshold;
receive a plurality of acceleration values from an accelerometer of the camera head;
calculate an average acceleration over a period of time before receiving the first electronic image; and
ascertain that the distal end of the endoscope is outside the body cavity when the number is above a predetermined region threshold and the average acceleration is below a predetermined movement threshold.

27. The endoscopic system of claim 20 wherein when the endoscopic console determines that the distal end of the endoscope is outside the body cavity, the endoscopic console is further configured to:
count a number of regions of the plurality of regions having values indicative of exposure below a predetermined exposure threshold;
create a scene indicator that a scene of the first electronic image is outside the body cavity; and
ascertain that the distal end of the endoscope is outside the body cavity when the number is above a predetermined region threshold and the scene indicator indicates the first electronic image is outside the body cavity.

28. The endoscopic system of claim 20 wherein the endoscopic console is further configured to, after reduction of the light provided to the endoscope:
receive a second electronic image from the camera head;
partition the second electronic image into a plurality of regions;
calculate a second value indicative of exposure for each region of the plurality of regions of the second electronic image, thereby creating a second plurality of values indicative of exposure;
determine that the distal end of the endoscope is within the body cavity, the determination based on the second plurality of values indicative of exposure; and
increase the light provided to the light port to a third illumination level higher than the second illumination level.
